(19) United States
(12) Patent Application Publication
(10) Pub. No.: US 2005/0143431 A1
(43) Pub. Date: Jun. 30, 2005

(54) Novel crystalline forms of parecoxib sodium

(75) Inventors: Reddy Bandi Parthasaradhi, Hyderabad (IN); Reddy Kura Rathnakar, Hyderabad (IN); Reddy Rapolu Raji, Hyderabad (IN); Reddy Dasari Muralidhara, Hyderabad (IN); Reddy Kesireddy Subadsh Chander, Hyderabad (IN)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: HETERO DRUGS LIMITED, Hyderabad, Andhrapradesh (IN)

(21) Appl. No.: 10/510,332

(22) PCT Filed: Apr. 4, 2003
(86) PCT No.: PCT/IN03/00140

(51) Int. Cl. C07D 261/06; A61K 31/42

(52) U.S. Cl. 514/378; 548/247

(57) ABSTRACT

The present invention relates to novel crystalline forms of parecoxib sodium, to processes for their preparation and to pharmaceutical compositions containing them.

### FIELD OF THE INVENTION

The present invention relates to novel crystalline forms of parecoxib sodium, to processes for their preparation and to pharmaceutical compositions containing them.

### BACKGROUND OF THE INVENTION

Parecoxib sodium of formula (1): 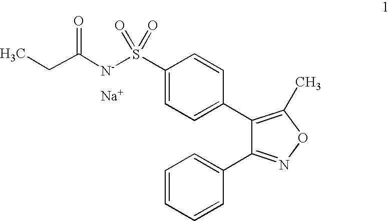
or N-[4-(5-Methyl-3-phenylisoxazol-4-yl)phenylsulfonyl]propionamide sodium salt is a highly selective and potent cyclooxygenase-2 inhibitor in human whole blood and useful in the treatment of arthritis and pain. The other therapeutic utilities of parecoxib and related compounds were disclosed in WO 9738986.

Crystalline forms of parecoxib sodium have not been reported in the literature. So, there is a need for stable polymorphs of parecoxib sodium for better pharmaceutical preparations.

We have discovered six stable novel crystalline forms of parecoxib sodium.

The object of the present invention is to provide stable novel crystalline forms of parecoxib sodium, processes for preparing these forms and pharmaceutical compositions containing them.

### DETAILED DESCRIPTION OF THE INVENTION

In accordance with the present invention, there is provided a novel crystalline form of parecoxib sodium (hereinafter referred to as parecoxib sodium form I), which is characterized by an x-ray powder diffraction pattern having peaks expressed as 2θ at about 5.7, 8.3, 10.4, 17.4, 21.0 and 23.2 degrees. FIG. 1 shows typical form I x-ray powder diffraction pattern.

In accordance with the present invention, there is provided a novel crystalline form of parecoxib sodium (hereinafter referred to as parecoxib sodium form II), which is characterized by an x-ray powder diffraction pattern having peaks expressed as 2θ at about 5.4, 6.8, 7.9, 10.6, 16.2, 17.1, 19.5, 20.4 and 22.4 degrees. FIG. 2 shows typical form II x-ray powder diffraction pattern.

In accordance with the present invention, there is provided a novel crystalline form of parecoxib sodium (hereinafter referred to as parecoxib sodium form III), which is characterized by an x-ray powder diffraction pattern having peaks expressed as 2θ at about 5.3, 5.9, 6.6, 7.8, 8.3, 10.7, 11.9, 12.2, 16.1, 19.5, 20.0, 21.6, 23.4 and 30.1 degrees. FIG. 3 shows typical form III x-ray powder diffraction pattern.

In accordance with the present invention, there is provided a novel crystalline form of parecoxib sodium (hereinafter referred to as parecoxib sodium form IV), which is characterized by an x-ray powder diffraction pattern having peaks expressed as 20 at about 5.2, 7.9, 12.1, 17.3, 17.9, 22.5, 23.4 and 27.1 degrees. FIG. 4 shows typical form IV x-ray powder diffraction pattern.

In accordance with the present invention, there is provided a novel crystalline form of parecoxib sodium (hereinafter referred to as parecoxib sodium form V), which is characterized by an x-ray powder diffraction pattern having peaks expressed as 20 at about 6.5, 7.7, 9.3, 10.6, 13.2, 15.5, 15.9, 17.4, 17.8, 20.2, 21.7, 22.1, 22.8, 23.4 and 24.3 degrees. FIG. 5 shows typical form V x-ray powder diffraction pattern.

In accordance with the present invention, there is provided a novel crystalline form of parecoxib sodium (hereinafter referred to as parecoxib sodium form VI), which is characterized by an x-ray powder diffraction pattern having peaks expressed as 2θ at about 5.4, 7.9, 9.5, 11.9, 18.1, 18.6, 20.9, 30.2 and 32.1 degrees. FIG. 6 shows typical form IV x-ray powder diffraction pattern.

In accordance with the present invention, there is provided processes for the preparation the novel forms I-VI of parecoxib sodium.

A process is provided for preparing parecoxib sodium form I from either parecoxib or parecoxib sodium. In this process either parecoxib sodium in any crystalline form or parecoxib and an sodium metal carrier are mixed with an alcohol solvent and then parecoxib sodium form I is isolated from the mixture.

Suitable alcohol solvents are methanol, ethanol, isopropyl alcohol, tert-butyl alcohol, n-butyl alcohol; and a mixture thereof. Preferred alcohol solvents are ethanol, methanol and isopropyl alcohol. Other solvents may also be mixed with the alcohol solvent as long as parecoxib form I can be isolated from the mixture.

A process is provided for preparing parecoxib sodium form II from either parecoxib or parecoxib sodium. In this process either parecoxib sodium in any crystalline form or parecoxib and an sodium metal carrier are mixed with acetonitrile and then parecoxib sodium form II is isolated from the mixture.

A process is provided for preparing parecoxib sodium form III from either parecoxib or parecoxib sodium. In this process either parecoxib sodium in any crystalline form or parecoxib and an sodium metal carrier are mixed with tetrahydrofuran and then parecoxib sodium form III is isolated from the mixture.

A process is provided for preparing parecoxib sodium form IV from either parecoxib or parecoxib sodium. In this process either parecoxib sodium in any crystalline form or parecoxib and an sodium metal carrier are mixed with an ether solvent and then parecoxib sodium form IV is isolated from the mixture.

Suitable ether solvents are diethyl ether, diisopropyl ether, methyl tert-butyl ether; and a mixture thereof.

A process is provided for preparing parecoxib sodium form V from either parecoxib or parecoxib sodium. In this process either parecoxib sodium in any crystalline form or parecoxib and an sodium metal carrier are mixed with an ester solvent and then parecoxib sodium form V is isolated from the mixture.

Suitable ester solvents are ethyl acetate (which is prererred), methyl acetate, isopropyl acetate, tert-butyl acetate, ethyl formate, methyl formate; and a mixture thereof.

A process is provided for preparing parecoxib sodium form VI from either parecoxib or parecoxib sodium. In this process either parecoxib sodium in any crystalline form or parecoxib and an sodium metal carrier are mixed with a ketone solvent and then parecoxib sodium form VI is isolated from the mixture.

Suitable ketone solvents are acetone (which is preferred), diethyl ketone, methyl ethyl ketone, methyl isobutyl ketone, methyl propyl ketone; and a mixture thereof.

Through out this specification, sodium metal carriers are sodium ethyl hexanoate, sodium hydroxide, and the like.

The mixing step of the processes of the present invention may be accomplished by, for example, slurrying or stirring. Isolation can be accomplished by, for example, filtration or centrifugation of the reaction mixture.

In accordance with the present invention, there is provided a pharmaceutical composition comprising parecoxib sodium form I and a pharmaceutically acceptable carrier or diluent.

In accordance with the present invention, there is provided a pharmaceutical composition comprising parecoxib sodium form II and a pharmaceutically acceptable carrier or diluent.

In accordance with the present invention, there is provided a pharmaceutical composition comprising parecoxib sodium form III and a pharmaceutically acceptable carrier or diluent.

In accordance with the present invention, there is provided a pharmaceutical composition comprising parecoxib sodium form IV and a pharmaceutically acceptable carrier or diluent.

In accordance with the present invention, there is provided a pharmaceutical composition comprising parecoxib sodium form V and a pharmaceutically acceptable carrier or diluent.

In accordance with the present invention, there is provided a pharmaceutical composition comprising parecoxib sodium form VI and a pharmaceutically acceptable carrier or diluent.

### BRIEF DESCRIPTION OF THE DRAWINGS

FIG. 1 is a x-ray powder diffraction spectrum of parecoxib sodium form I.

FIG. 2 is a x-ray powder diffraction spectrum of parecoxib sodium form II.

FIG. 3 is a x-ray powder diffraction spectrum of parecoxib sodium form 111.

FIG. 4 is a x-ray powder diffraction spectrum of parecoxib sodium form IV.

FIG. 5 is a x-ray powder diffraction spectrum of parecoxib sodium form V.

FIG. 6 is a x-ray powder diffraction spectrum of parecoxib sodium form VI.

x-Ray powder diffraction spectrum was measured on a Siemens D5000 x-ray powder diffractometer having a copper-Kα radiation.

The invention will now be further described by the following examples, which are illustrative rather than limiting.

### EXAMPLE 1

Parecoxib (5.0 gm) is dissolved in ethanol (25 ml) and then sodium hydroxide (0.5 gm) is added. The contents are maintained for 2 hours at 25° C. to 30° C., cooled to 0° C. and the separated crystals are collected by filtration to give 4.5 gm of parecoxib sodium form I.

### EXAMPLE 2

Parecoxib (5.0 gm) is dissolved in acetonitrile (25 ml) and then sodium hydroxide (0.5 gm) is added. The contents are maintained for 2 hours 30 minutes at 25° C. to 27° C., cooled to 0° C. and the separated crystals are collected by filtration to give 4.0 gm of parecoxib sodium form II.

### EXAMPLE 3

Parecoxib sodium form II (10 gm) is mixed with isopropyl alcohol (50 ml), the contents are maintained for 2 hours at 25° C. to 30° C., cooled to 0° C. and solid is collected by filtration to give parecoxib sodium form I in quantitative yield.

### EXAMPLE 4

Parecoxib (5.0 gm) is dissolved in tetrahydrofuran (30 ml) and then sodium hydroxide (0.5 gm) is added. The contents are maintained for 1 hours 30 minutes at 20° C. to 25° C., cooled to 0° C. and the separated crystals are collected by filtration to give 3.0 gm of parecoxib sodium form III.

### EXAMPLE 5

Parecoxib sodium form I (10 gm) is mixed with tetrahydrofuran (60 ml), the contents are stirred for 3 hours at 25° C. to 30° C., cooled to 0° C. and solid is collected by filtration to give 9.5 gm of parecoxib sodium form III.

### EXAMPLE 6

Parecoxib (5.0 gm), methyl tert-butyl ether (25 ml) and sodium hydroxide (0.5 gm) are mixed. The contents are maintained for 3 hours at 28° C. to 30° C., cooled to 20° C. and the separated crystals are collected by filtration to give 4.5 gm of parecoxib sodium form IV.

### EXAMPLE 7

Parecoxib (5.0 gm) is dissolved in ethyl acetate (30 ml) and then sodium hydroxide (0.5 gm) is added. The contents are maintained for 18 hours at 28° C. to 30° C. The separated crystals are collected by filtration to give 4.0 gm of parecoxib sodium form V.

### EXAMPLE 8

Parecoxib sodium form II (5 gm) is mixed with ethyl acetate (25 ml), the contents are maintained for 2 hours at 25° C. to 30° C., cooled to 0° C. and solid is collected by filtration to give 4.8 gm of parecoxib sodium form V.

### EXAMPLE 9

Parecoxib (5.0 gm) is dissolved in acetone (25 ml) and then sodium hydroxide (0.5 gm) is added. The contents are maintained for 2 hours at 57° C. to 60° C., cooled to 25° C. and the separated crystals are collected by filtration to give 4.0 gm of parecoxib sodium form VI.

1. A crystalline parecoxib sodium form I, characterized by an x-ray powder diffraction pattern having peaks expressed as 2θ at about 5.7, 8.3, 10.4, 17.4, 21.0 and 23.2 degrees.

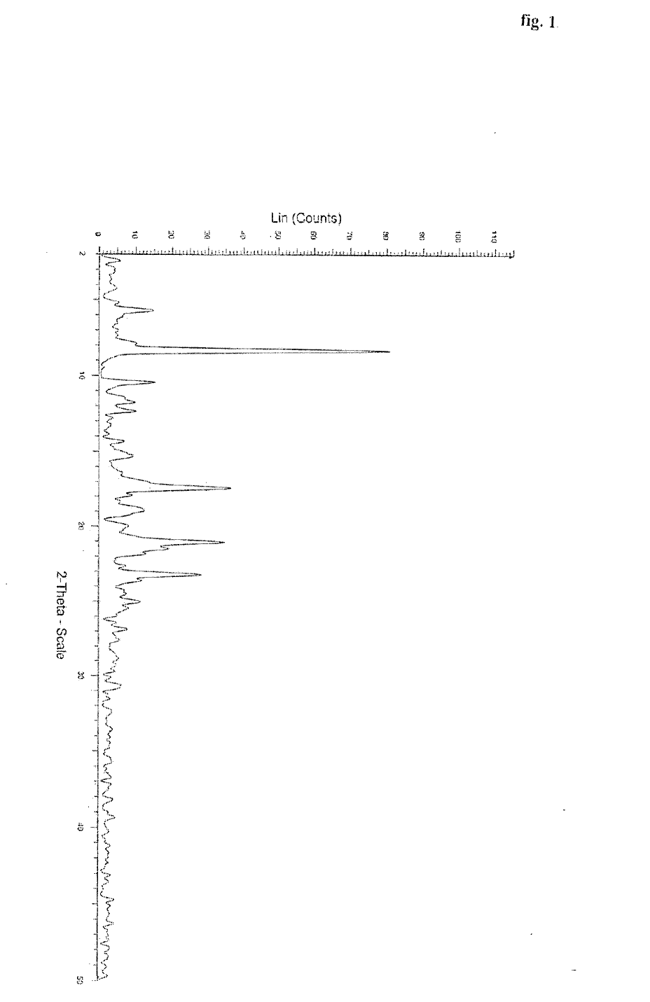

2. The crystalline parecoxib sodium form I as defined in claim 1, further characterized by an x-ray powder diffraction pattern as in FIG. 1.

3. A crystalline parecoxib sodium form II, characterized by an x-ray powder diffraction pattern having peaks expressed as 2θ at about 5.4, 6.8, 7.9, 10.6, 16.2, 17.1, 19.5, 20.4 and 22.4 degrees.

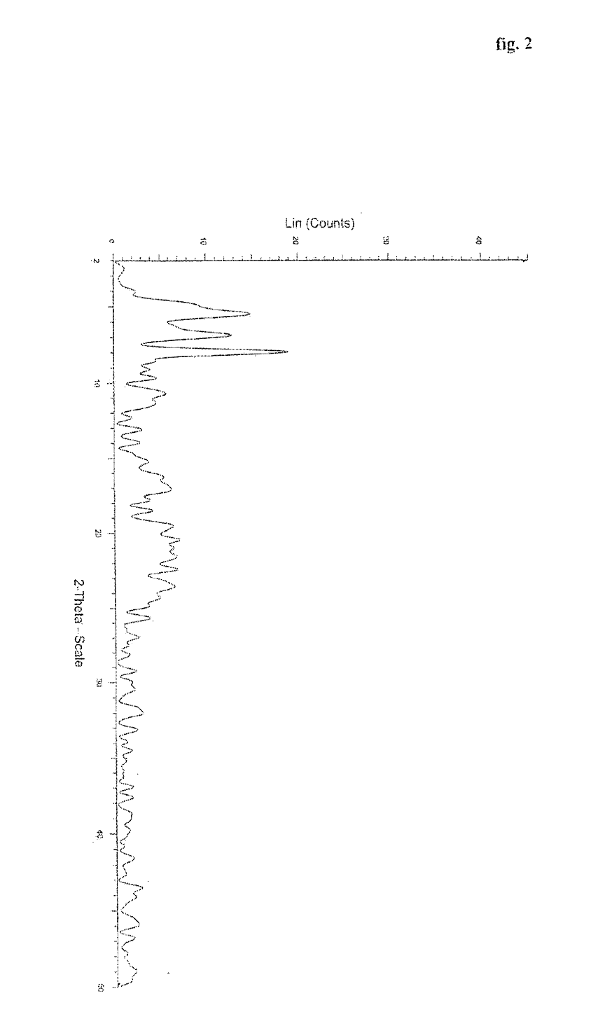

4. The crystalline parecoxib sodium form II as defined in claim 3, further characterized by an x-ray powder diffraction pattern as in FIG. 2.

5. A crystalline parecoxib sodium form III, characterized by an x-ray powder diffraction pattern having peaks expressed as 2θ at about 5.3, 5.9, 6.6, 7.8, 8.3, 10.7, 11.9, 12.2, 16.1, 19.5, 20.0, 21.6, 23.4 and 30.1 degrees.

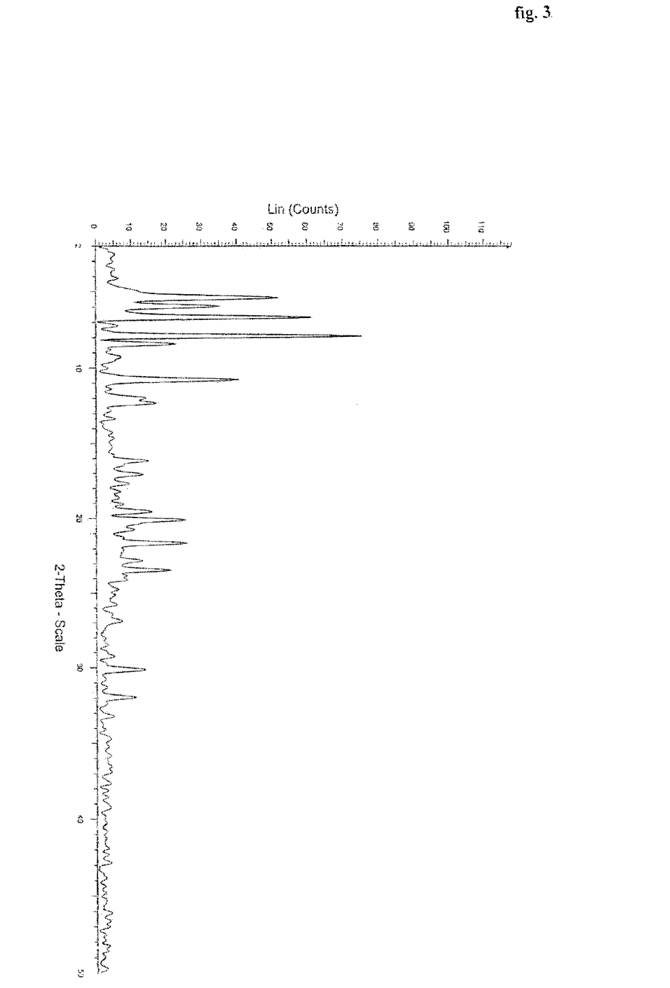

6. The crystalline parecoxib sodium form III as defined in claim 5, further characterized by an x-ray powder diffraction pattern as in FIG. 3.

7. A crystalline parecoxib sodium form IV, characterized by an x-ray powder diffraction pattern having peaks expressed as 2θ at about 5.2, 7.9, 12.1, 17.3, 17.9, 22.5, 23.4 and 27.1 degrees.

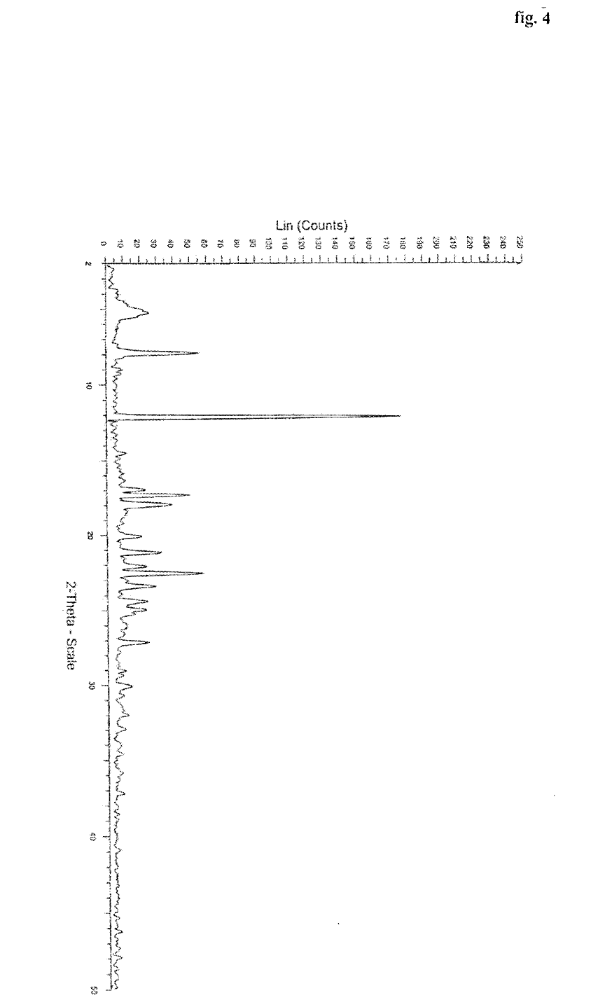

8. The crystalline parecoxib sodium form IV as defined in claim 7, further characterized by an x-ray powder diffraction pattern as in FIG. 4.

9. A crystalline parecoxib sodium form V, characterized by an x-ray powder diffraction pattern having peaks expressed as 2θ at about 6.5, 7.7, 9.3, 10.6, 13.2, 15.5, 15.9, 17.4, 17.8, 20.2, 21.7, 22.1, 22.8, 23.4 and 24.3 degrees.

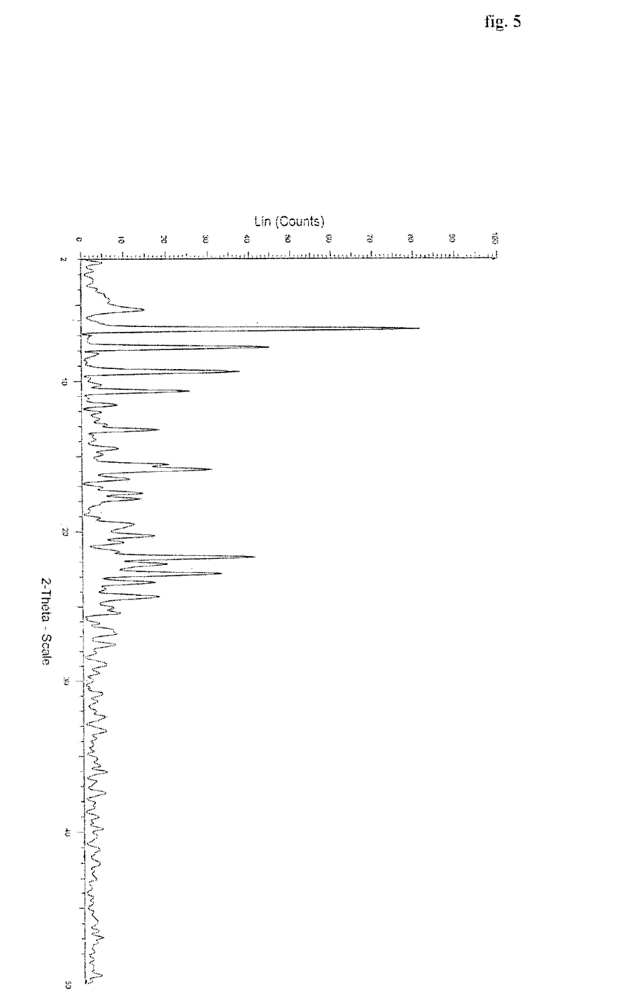

10. The crystalline parecoxib sodium form V as defined in claim 9, further characterized by an x-ray powder diffraction pattern as in FIG. 5.

11. A crystalline parecoxib sodium form VI, characterized by an x-ray powder diffraction pattern having peaks expressed as 2θ at about 5.4, 7.9, 9.5, 11.9, 18.1, 18.6, 20.9, 30.2 and 32.1 degrees.

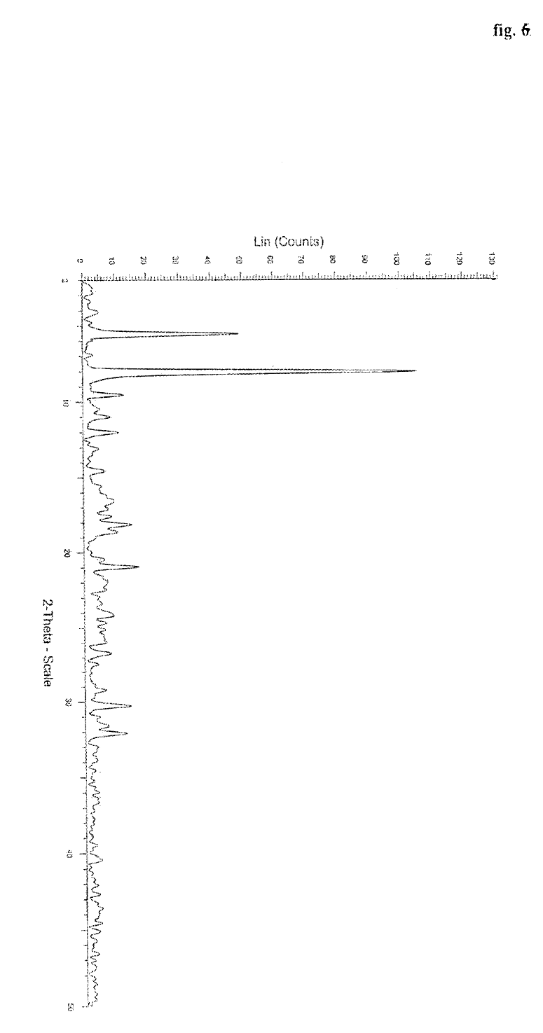

12. The crystalline parecoxib sodium form VI as defined in claim 11, further characterized by an x-ray powder diffraction pattern as in FIG. 6.

13. A process for preparing parecoxib sodium form I as defined in claim 1, which comprises the steps of:
a) mixing together i) either 1) parecoxib sodium or 2) parecoxib and a sodium metal carrier, and
ii) an alcohol solvent; and
b) isolating parecoxib sodium form I from the mixture;
wherein the alcohol solvent is selected from the group consisting of methanol, ethanol, isopropyl alcohol, tert-butyl alcohol and n-butyl alcohol.

14. The process according to claim 13, wherein the sodium metal carrier is sodium hydroxide.

15. The process according to claim 13, wherein the alcohol solvent is ethanol.

16. A process for preparation of parecoxib sodium form II as defined in claim 3, which comprises the steps of:
a) mixing together i) either 1) parecoxib sodium or 2) parecoxib and a sodium metal carrier, and
ii) acetonitrile; and
b) isolating parecoxib sodium form II from the mixture.

17. The process according to claim 16, wherein sodium metal carrier is sodium hydroxide.

18. A process for preparation of parecoxib sodium form III as defined in claim 5, which comprises the steps of:
a) mixing together i) either 1) parecoxib sodium or 2) parecoxib and a sodium metal carrier, and
ii) tetrahydrofuran; and
b) isolating parecoxib sodium form III from the mixture.

19. The process according to claim 18, wherein the sodium metal carrier is sodium hydroxide.

20. A process for preparation of parecoxib sodium form IV as defined in claim 7, which comprises the steps of:
a) mixing together i) either 1) parecoxib sodium or 2) parecoxib and a sodium metal carrier, and
ii) an ether solvent; and
b) isolating parecoxib sodium form IV from the mixture;
wherein the ether solvent is selected from the group consisting of diethyl ether, diisopropyl ether and methyl tert-butyl ether.

21. The process according to claim 20, wherein sodium metal carrier is sodium hydroxide.

22. The process according to claim 20, wherein the ether solvent is methyl tert-butyl ether.

23. A process for preparation of parecoxib sodium form V as defined in claim 9, which comprises the steps of:
a) mixing together i) either 1) parecoxib sodium or 2) parecoxib and a sodium metal carrier, and
ii) an ester solvent; and
b) isolating parecoxib sodium form V from the mixture;
wherein the ester solvent is selected from the group consisting of ethyl acetate, methyl acetate, isopropyl acetate, tert-butyl acetate, ethyl formate and methyl formate.

24. The process according to claim 23, wherein sodium metal carrier is sodium hydroxide.

25. The process according to claim 23, wherein the ether solvent is ethyl acetate.

26. A process for preparation of parecoxib sodium form VI as defined in claim 11, which comprises the steps of:
a) mixing together i) either 1) parecoxib sodium or 2) parecoxib and a sodium metal carrier, and
ii) a ketone solvent; and
b) isolating parecoxib sodium form VI from the mixture;
wherein the ketone solvent is selected from the group consisting of acetone, diethyl ketone, methyl ethyl ketone, methyl isobutyl ketone and methyl propyl ketone.

27. The process according to claim 26, wherein sodium metal carrier is sodium hydroxide.

28. The process according to claim 26, wherein the ketone solvent is acetone.

29. The process according to claim 13, wherein parecoxib sodium is selected from the group consisting of form II of claim 3, form III of claim 5, form IV of claim 7, form V of claim 9 and form VI of claim 11.

30. The process according to claim 16, wherein parecoxib sodium is selected from the group consisting of form I of claim 1, form III of claim 5, form IV of claim 7, form V of claim 9 and form VI of claim 11.

31. The process according to claim 18, wherein parecoxib sodium is selected from the group consisting of form I of claim 1, form II of claim 3, form IV of claim 7, form V of claim 9 and form VI of claim 11.

32. The process according to claim 20, wherein parecoxib sodium is selected from the group consisting of form I of claim 1, form II of claim 3, form III of claim 5, form V of claim 9 and form VI of claim 11.

33. The process according to claim 23, wherein parecoxib sodium is selected from the group consisting of form I of claim 1, form II of claim 3, form III of claim 5, form IV of claim 7 and form VI of claim 11.

34. The process according to claim 26, wherein parecoxib sodium is selected from the group consisting of form I of claim 1, form II of claim 3, form III of claim 5, form IV of claim 7 and form V of claim 9.

35. A pharmaceutical composition comprising parecoxib sodium form I of claim 1 and a pharmaceutically acceptable carrier or diluent.

36. A pharmaceutical composition comprising parecoxib sodium form II of claim 3 and a pharmaceutically acceptable carrier or diluent.

37. A pharmaceutical composition comprising parecoxib sodium form III of claim 5 and a pharmaceutically acceptable carrier or diluent.

38. A pharmaceutical composition comprising parecoxib sodium form IV of claim 7 and a pharmaceutically acceptable carrier or diluent.

39. A pharmaceutical composition comprising parecoxib sodium form V of claim 9 and a pharmaceutically acceptable carrier or diluent.

40. A pharmaceutical composition comprising parecoxib sodium form VI of claim 11 and a pharmaceutically acceptable carrier or diluent.
